(19) United States
(12) Patent Application Publication
(10) Pub. No.: US 2025/0040829 A1
(43) Pub. Date: Feb. 6, 2025

(54) METHOD FOR DETECTING HELICOBACTER PYLORI USING AN ELECTROCHEMICAL TEST STRIP

(71) Applicant: UltraE Co. Ltd., Taichung City (TW)

(72) Inventors: Hsieh-Hsun Chung, Taichung City (TW); Ping-Hsi Hsieh, Taichung City (TW)

(21) Appl. No.: 18/238,276

(22) Filed: Aug. 25, 2023

(30) Foreign Application Priority Data
Aug. 3, 2023 (TW) 112129172

(51) Int. Cl. A61B 5/08; A61B 5/097; G01N 27/30

(52) U.S. Cl. CPC A61B 5/082; A61B 5/097; G01N 27/302

(57) ABSTRACT

A method for detecting Helicobacter pylori using an electrochemical test strip, comprising the following steps: making a subject to exhale a first gas sample into a detection zone of an electrochemical test strip via a blowpipe, using the electrochemical test strip to detect the first gas sample to obtain a background pH data; the subject is allowed to consume urea; making the subject to exhale a second gas sample into the detection zone of another/the electrochemical test strip via the blowpipe, using the electrochemical test strip to detect the second gas sample to obtain a desired pH data; comparing the background pH data and the desired pH data to determine whether the subject's stomach is infected with Helicobacter pylori.

### BACKGROUND OF THE INVENTION

### 1. Technical Field

The present invention relates to a method for detecting Helicobacter pylori, and more particularly to a method for detecting Helicobacter pylori using an electrochemical test strip.

### 2. Description of Related Art

Helicobacter pylori has been found to be involved in the pathogenesis of common gastrointestinal diseases (eg gastritis, gastric ulcer, duodenal ulcer), and infection with Helicobacter pylori is also considered to be a possible risk factor for gastric cancer.

In the prior art, the clinical diagnosis method of Helicobacter pylori is roughly divided into two categories: invasive detection and non-invasive detection. The invasive detection needs to use gastroscope, which often makes patients feel uncomfortable. Besides, Helicobacter pylori is often scattered in the stomach in the form of plaques, so it is easy to cause false negative results due to mistakes in specimen collection.

On the other hand, carbon-13 urea breath test is a common non-invasive method for detecting Helicobacter pylori. The carbon-13 urea used in this test is labeled with a carbon-13 isotope in the urea molecule. After the subject eats the carbon-13 urea, the carbon-13 urea will be converted into carbon dioxide with a carbon-13 isotope (carbon-13 dioxide) if there is Helicobacter pylori in the stomach. The carbon-13 dioxide can then be exhaled from the body and be collected. By measuring the amount of carbon-13 dioxide, one can determine whether the subject is infected with Helicobacter pylori.

More specifically, the carbon-13 urea breath test collects exhaled gas samples before and after the subject consumes carbon-13 urea, and then the two samples are put into an isotope mass spectrometer to determine the amount of carbon-13 isotope. By comparing the measured values, whether the subject is infected with Helicobacter pylori can be determined.

However, the carbon-13 urea breath test has its shortcomings in that it usually takes several days for clinical subjects to obtain the test results, large professional equipment is required to perform the measurement, and carbon-13 urea is not easy to obtain and its price is relatively high.

### BRIEF SUMMARY OF THE INVENTION

The primary objective of the present invention is to provide a method that can quickly determine the presence of Helicobacter pylori in the stomach of a subject without the use of large professional equipment.

To achieve the foregoing and other objectives, the present invention provides a method for detecting Helicobacter pylori using an electrochemical test strip, in which:

The electrochemical test strip includes a strip body, a working electrode, a pH sensing layer, a reference electrode, a solid water absorption layer, a battery, a controller, a casing and a blowpipe. The strip body has a detection area for contact with a gas sample. The working electrode is disposed on the strip body and has a first part located in the detection area. The pH sensing layer is disposed on the first part of the working electrode located in the detection area. The reference electrode is disposed on the strip body and has a second part located in the detection area. The solid water absorption layer is disposed in the detection area and covers the pH sensing layer and the second part. The solid water absorption layer is adapted to absorb or adsorb water in the gas sample in a manner that the first and second parts are electrically connected to each other. The controller electrically connected to the working electrode, the reference electrode and the battery. The casing covers the strip body. The blowpipe is disposed on the casing and has a flow channel for the gas sample to flow to the detection area.

The method for detecting Helicobacter pylori comprises the following steps:
filling the detection area with a first gas sample by making a subject blow on the blowpipe of the electrochemical test strip, and detecting the first gas sample with the electrochemical test strip to obtain a background pH data;
feeding the subject with urea;
filling the detection area with a second gas sample by making the subject blow on the blowpipe of the same or another said electrochemical test strip, and detecting the second gas sample with the electrochemical test strip to obtain a desired pH data; and
comparing the background pH data and the desired pH data to determine whether the subject's stomach has Helicobacter pylori.

Other effects and features of the present invention will be described in detail hereinbelow.

### BRIEF DESCRIPTION OF THE SEVERAL VIEWS OF THE DRAWINGS

FIG. 1 is a perspective view of a first embodiment of the present invention;

FIG. 2 is a 2-2 profile of FIG. 1;

FIG. 3 is a 3-3 profile of FIG. 1;

FIGS. 4 and 5 show applications of the first embodiment of the present invention;

FIG. 6 is an explosive drawing of a second embodiment of the present invention;

FIG. 7 is an explosive drawing of the second embodiment of the present invention in which the circuit board is shown at a different angle;

FIG. 8 is a drawing showing a tablet computer usable with the second embodiment of the present invention;

FIG. 9 shows an application of the second embodiment of the present invention;

FIG. 9A is a drawing showing a gas sampling bag of a third embodiment of the present invention;

FIG. 9B is a drawing showing a gas sampling bag of a fourth embodiment of the present invention;

FIGS. 10-13 are graphs of experimental results of the gas pH measurement of the present invention;

FIGS. 14-19 are graphs of experimental results that the present invention is applied to detect Helicobacter pylori, in which the thinner line represents a background pH data and the thicker line represents a desired pH data.

FIG. 20 is a electrochemical test strip of a fifth embodiment of the present invention;

FIG. 21 shows an application of the fifth embodiment of the present invention;

FIGS. 22-24 are graphs of experimental results that the electrochemical test strip of the fifth embodiment of the present invention is applied to detect Helicobacter pylori, in which the thinner line represents a background pH data and the thicker line represents a desired pH data.

### DETAILED DESCRIPTION OF THE INVENTION

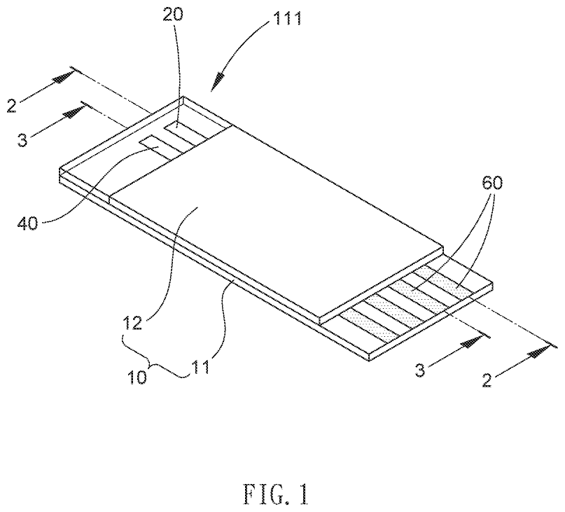

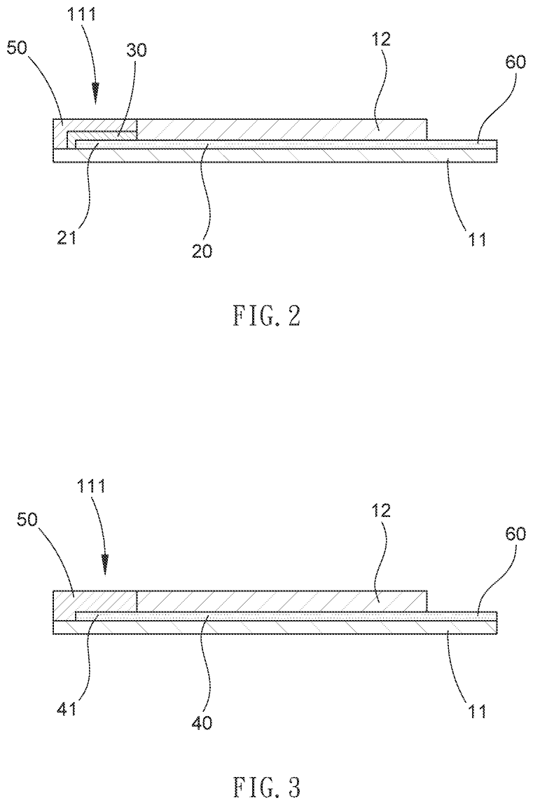

Referring to FIGS. 1-2 for an electrochemical test strip of the first embodiment of the present invention. The electrochemical test strip has a strip body 10, a working electrode 20, a pH sensing layer 30, a reference electrode 40, a solid water absorption layer 50 and a plurality of gold fingers 60. The electrochemical test strip of the present invention can be used to measure pH values of gas samples.

The strip body 10 includes a substrate 11 and a protection layer 12 and has a detection area 111. The working electrode 20, the reference electrode 40 and the gold fingers 60 is disposed on the substrate 11 of the strip body 10. The protection layer 12 is disposed on the substrate 11 and covers a part of the working electrode 20 and a part of the reference electrode 40. Both distal ends of the substrate 11 are not covered by the protection layer 12. One of the distal ends constitute the detection area 111 and can be in contact with gas samples. The other of the distal ends exposes the gold fingers 60, which means the gold fingers 60 is not located in the detection area 111. The working electrode 20 has a first part 21 located in the detection area 111, and the reference electrode 40 has a second part 41 located in the detection area 111 as well. The gold fingers 60 are electrically connected to the working electrode 20 and the reference electrode 40, respectively. Some of the gold fingers 60 may be electrically connected to other electrodes disposed on the strip body 10, if any.

The pH sensing layer 30 covers and is formed on the first part 21 of the working electrode 20. The pH sensing layer 30 may be made of pH sensing materials such as aniline compounds (such as aniline and clenbuterol), aromatic heterocyclic compounds (such as melamine, lamotrigine and altretamine), aminophenols (such as acetaminophen), metal oxides (such as copper oxide, iridium oxide), azo compounds (such as azobenzene) or conductive polymers (such as Nafion and polypyrrole). These pH sensing materials have bonding sites which are adsorbable/desorbable with hydrogen ions. When these bonding sites adsorb/desorb hydrogen ions, their chemical potentials will vary.

The solid water absorption layer 50 is located in the detection area 111 and covers the pH sensing layer 30 and the second part 41 of the reference electrode 40. The solid water absorption layer 50 is adapted to absorb or adsorb water in the gas sample (water vapor) so that the first and second parts 21 and 41 can be electrically connected to each other. The solid water absorption layer 50 can be, but not limited to, water-absorbing surfactants, humectants, antioxidants, thickeners, conductive polymers or mixtures thereof. The water-absorbing surfactants can be cetyltrimethyl ammonium chloride (CTAC). The humectants can be sorbitol, PCA-Na, 1,3-propanediol, co-enzyme Q10, ceramide, allantoin, collagen, hyaluronic acid, urea, glycerin or polyethylene glycol. The antioxidants can be Vitamin C or 3,4-dihydroxy-cinnamic acid. The thickeners can be methylcellulose, carboxylmethyl cellulose sodium salt (CMC) or polyvinyl alcohol. The conductive polymers can be Nafion or poly acryl sodium (PAS). In a single electrochemical test strip, the pH sensing layer 30 and the solid water absorption layer 50 are made of different materials.

When measuring pH of gas, the electrochemical test strip can be brought in contact with the gas sample. The solid water absorption layer 50 will start to absorb/adsorb water (in the form of water vapor for instance). The absorbed/adsorbed water plays two roles, one of which is to enable the working electrode 20 and the reference electrode 40 to electrically connect to each other, while the other of which is to dynamically adsorb/desorb water-soluble pH substances originally contained in the gas test sample in vaporized form, if any, until the adsorption-desorption equilibrium is reached. The adsorbed water-soluble pH substances can interact with the pH sensing layer 30, generating a potential difference between the working electrode and the reference electrode, based on which the pH vale of the gas sample can be measured. The water-soluble pH substances can be, but not limited to, ammonia, nitric oxide, nitrogen dioxide, hydrochloric acid, acetic acid or other organic acids.

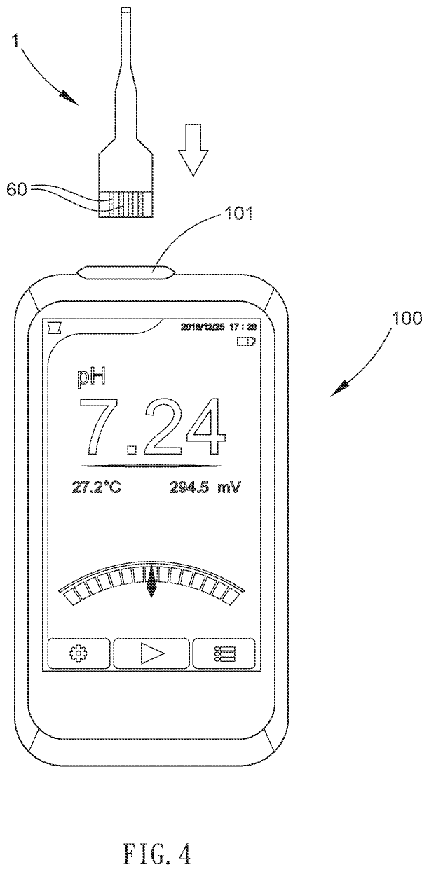

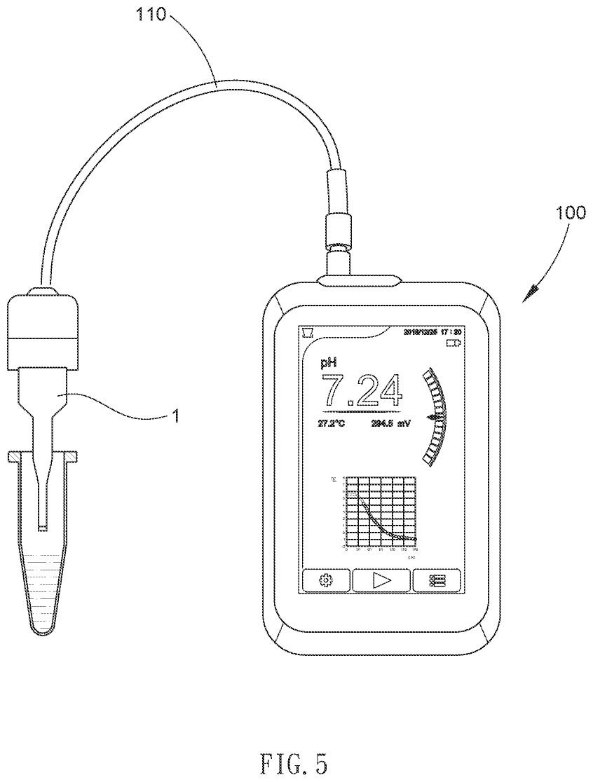

Please refer to FIG. 4. The electrochemical test strip 1 can be used with a detector 100, which has a connection port 101 for the test strip 1 to insert therein. The connection port is formed with electrical contacts (not shown) capable of electrically connected to the gold fingers 60 so as to perform pH measurement. Alternatively, as shown in FIG. 5, the detector 100 can be provided with an extension adapter 110 for the electrochemical test strip 1 to electrically connect thereto so as to perform pH measurement.

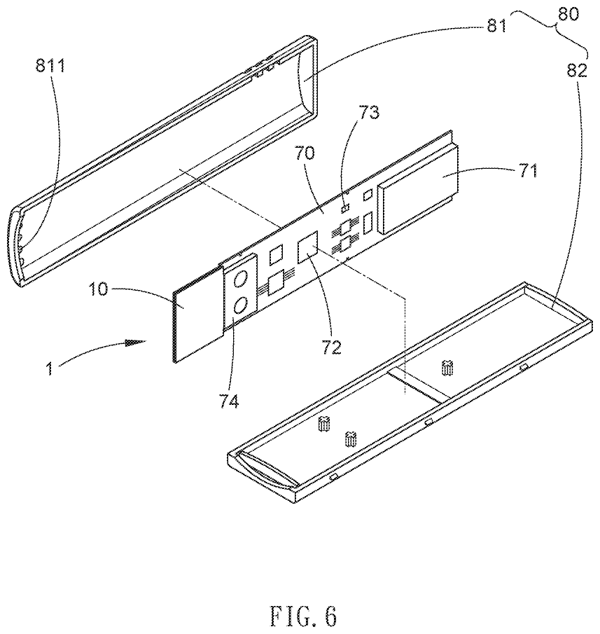

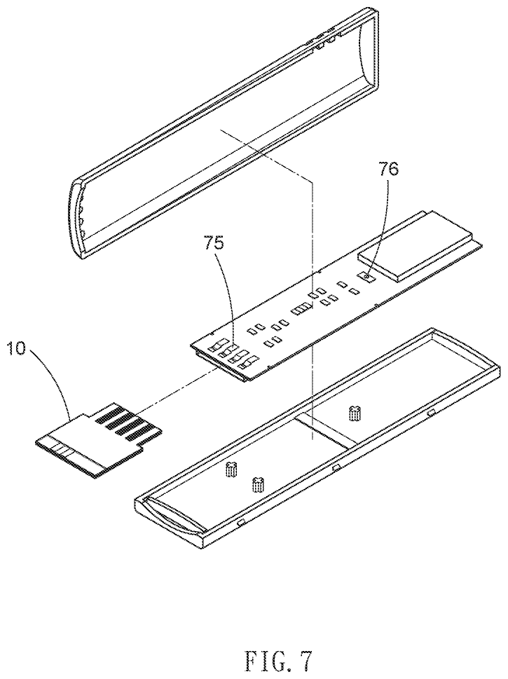

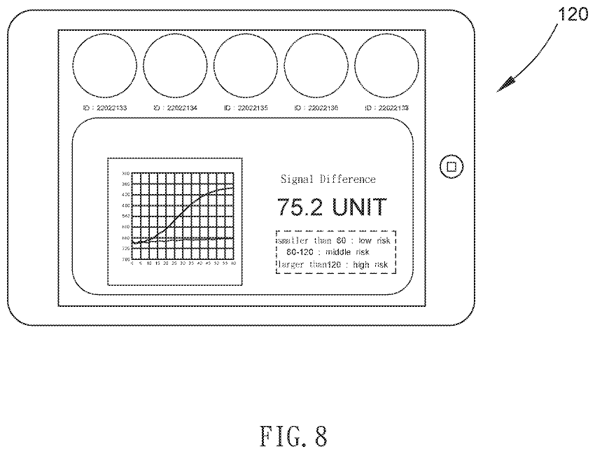

Please refer to FIGS. 6 and 7 for the electrochemical test strip of the second embodiment of the present invention. In addition to the parts shown in the first embodiment, the electrochemical test strip further includes a circuit board 70 and a casing 80. The circuit board 70 has a battery 71, a controller 72, an antenna 73, a strip slot 74, a plurality of strip contacts 75 and a switch 76, in which the battery 71, the antenna 73 and the strip contacts 75 are electrically connected to the controller 72, respectively. The antenna 73 is adapted to form a wireless signal connection with an external device, which can be a tablet computer 120 shown in FIG. 8 or other devices such as mobile phones and computers which can form a wireless connection with the electrochemical test strip 1 and can display, process and/or relay detection parameter signals. The strip slot 74 is adapted for the strip body 10 to insert therein. The strip contacts 75 can be, for example, jack strips. A part of every strip contact 75 is located in the strip slot 74 so that the gold fingers 60 of the strip body 10 can form electrical connection with the strip contacts 75, respectively. The switch 76 is adapted for controlling on/off circuit between the battery 71 and the controller 72. The casing 80 has a first casing part 81 and a second casing part 82 for encapsulating the strip body 10 and the circuit board 70. The first casing part 81 of the casing 80 has a plurality of air holes adapted for the gas sample to flow into the detection area.

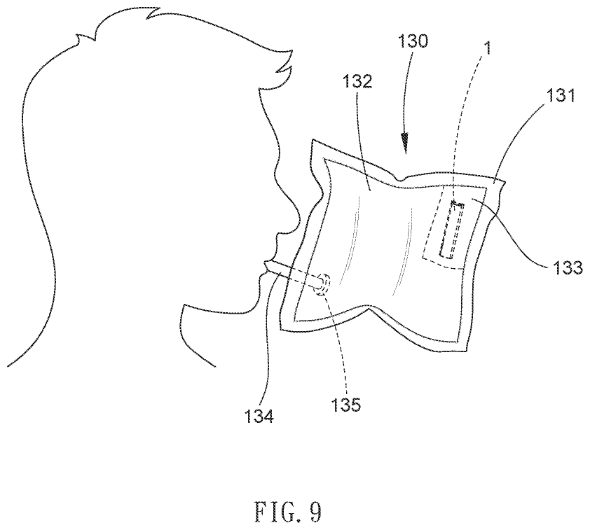

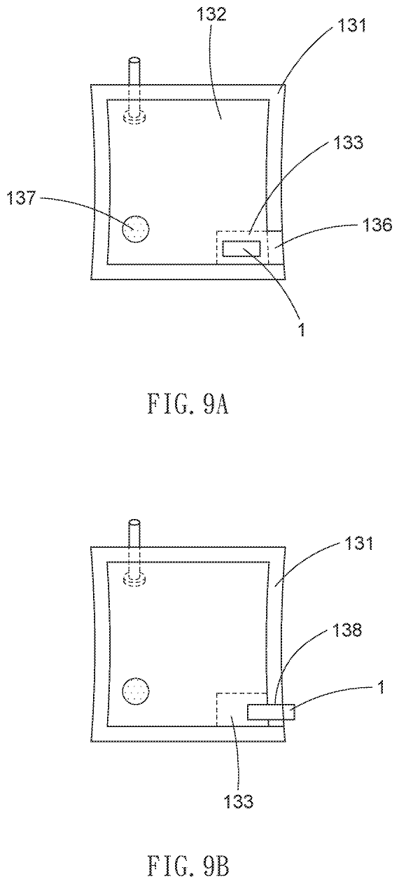

The electrochemical test strip of the second embodiment can perform the measurement of the potential difference between the working electrode and the reference electrode without the need the detectors 100 shown in FIGS. 4 and 5. The electrochemical test strip 1 of the second embodiment can be placed in a gas sampling bag 130 as shown in FIG. 9 to perform pH measurement and then transmit detection results to the device as shown in FIG. 8.

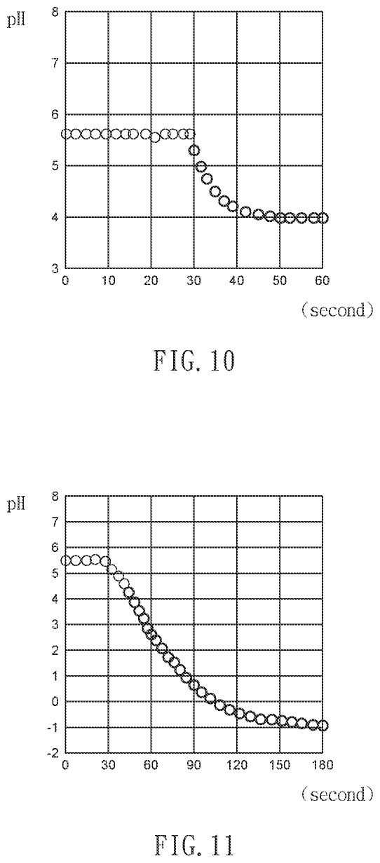

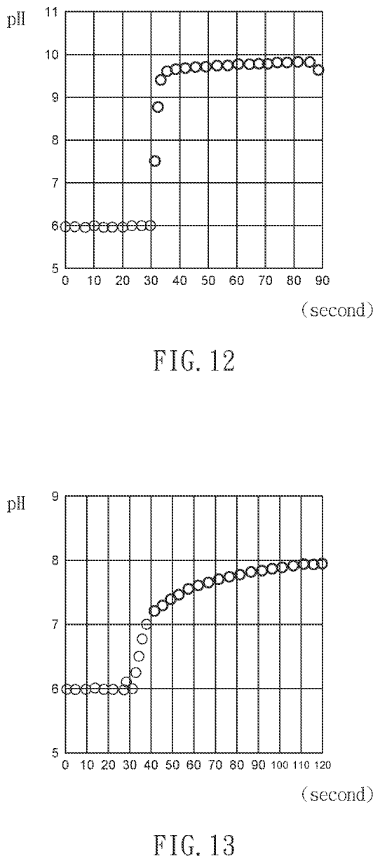

To verify the performance of the electrochemical test strip of the present invention, the following tests were conducted. Four samples, including 10% acetic acid aqueous solution, 10% hydrochloric acid aqueous solution, 5% ammonia aqueous solution and fish meat (stale meat can produce ammonia gas), were prepared. The electrochemical test strips were used to detect the volatile gases of the samples, individually, without direct contact with the samples. The test results are shown in FIGS. 10-13. According to the results, pH changes were not detected for a period of time at the beginning of the tests because the solid water absorption layer of each test strip just started absorbing/adsorbing water (in the form of water vapor) in the volatile gases and the absorbed/adsorbed water also took some time to gradually adsorb pH substances. After a short while, pH changes were noticeable and gradually became stable because the adsorption-desorption equilibrium was met between the water and the pH substances. The results show that the electrochemical test strips of the present invention can indeed detect whether there are pH substances in the gas samples and can reflect acidity/alkalinity of the gas samples.

A method for detecting Helicobacter pylori using the electrochemical test strip is described as follows. Helicobacter pylori can survive in the human stomach and can secrete urea-decomposing enzymes to convert urea into carbon dioxide and ammonia. The converted carbon dioxide and ammonia can be excreted with exhalation, in which the ammonia can be detected with the electrochemical test strip of the present invention. The method includes the following steps:
as shown in FIG. 9, filling the gas sampling bag 130 with a first gas sample blown by a subject, and detecting the first gas sample with the electrochemical test strip 1 to obtain a background pH data: in order to mitigate test errors, the subject can fast for a period of time before the test: in the present embodiment, the gas sampling bag 130 includes a bag body 131, which has a main sampling chamber 132, a electrochemical test strip accommodating chamber 133, a blowpipe 134 and an oneway valve 135; the blowpipe 134 has a gas channel in communication with the main sampling chamber 132; the oneway valve 135 is disposed on the blowpipe 134 so that the gas sample is only allowed to flow in a direction from the gas channel to the main sampling chamber 132; the electrochemical test strip accommodating chamber 133 is in communication with the main sampling chamber 132 and is adapted to accommodate the electrochemical test strip 1: in the embodiment shown in FIG. 9, the electrochemical test strip 1 is embedded in the bag body 131; in the embodiments shown in FIGS. 9A and 9B, the electrochemical test strip 1 is replaceable; as shown in FIG. 9A, the bag body 131 has a selectively opened or closed pocket opening 136 in communication with the electrochemical test strip accommodating chamber 133: when the pocket opening 136 is opened, the electrochemical test strip 1 inside the bag body 131 can be replaced: in addiction, the bag body 131 further has a bleed valve 137 adapted to release the gas sample from the main sampling chamber 132, so that a single gas sampling bag 130 can be used for multiple times: as shown in FIG. 9B, the bag body 131 has a electrochemical test strip slot 138 in communication with the electrochemical test strip accommodating chamber 133; the electrochemical test strip slot 138 is adapted for the electrochemical test strip 1 to insert therein so that a part of the electrochemical test strip 1 is accommodated in the electrochemical test strip accommodating chamber 133: preferably, when the electro chemical test strip 1 is inserted into the electrochemical test strip slot 138 the electrochemical test strip 1 can make the electrochemical test strip slot 138 airtight: in other possible embodiments, the exposed part of the electrochemical test strip 1 can be mechanically connected to a detector (not shown);
then, feeding the subject with urea and waiting for a period of time, such as 15 minutes or longer;
then, filling the gas sampling bag 130 with a second gas sample blown by the subject, and detecting the second gas sample with the electrochemical test strip 1 to obtain a desired pH data: the subject can use the same gas sampling bag 130 and the same electrochemical test strip 1 as the first sampling: alternatively, the subject can use a different gas sampling bag 130 and/or a different electrochemical test strip 1 if desired: if the same gas sampling bag 130 is used in the two samplings, the first gas sample needs to be released from the gas sampling bag 130 before the second sampling;
lastly, comparing the background pH data and the desired pH data to determine whether the subject's stomach has Helicobacter pylori.

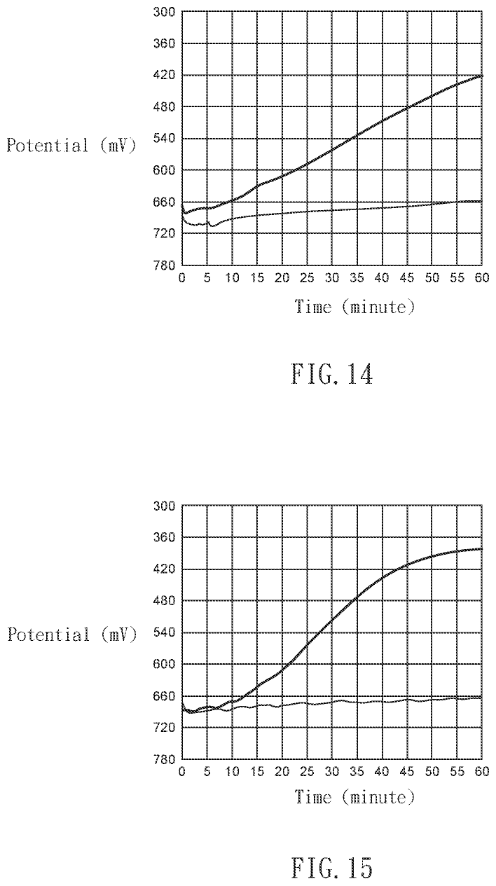

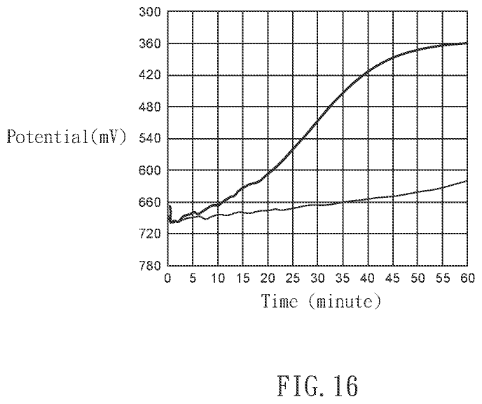

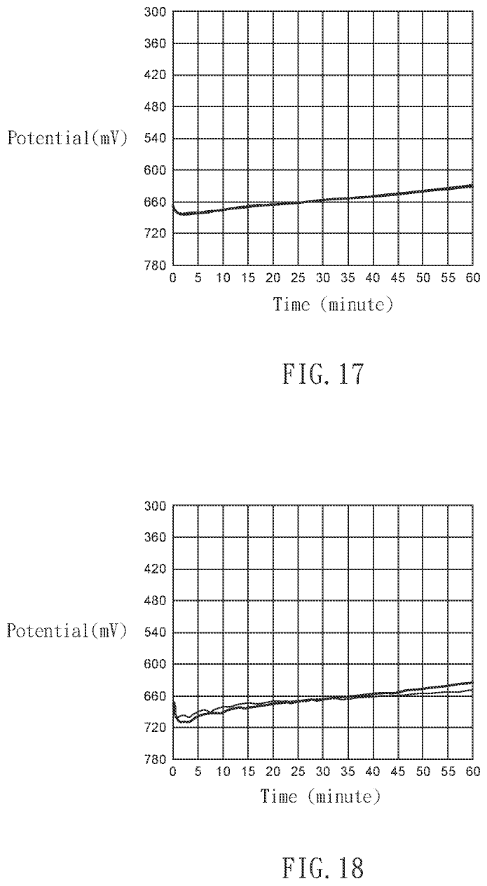

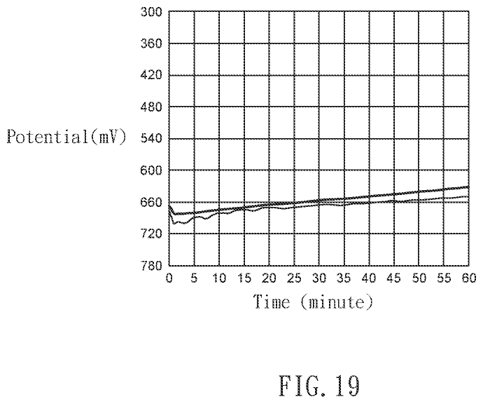

The applicant applied the aforementioned method on six subjects to determine whether the subjects were infected with Helicobacter pylori. In addition to the aforementioned method, all the subjects also took a conventional test for Helicobacter pylori (Carbon-13 Urea Breath Test) in a medical institution, in which three of them were found positive and the others negative. FIGS. 14-19 show the results of the aforementioned method performed by the six subjects. FIGS. 14-16 are the results of the three subjects who were found positive in the conventional test for Helicobacter pylori, in which there are obvious differences between the background pH data and the desired pH data and there are noticeable potential differences between the two data after a period of time. On the other hand, FIGS. 17-19 are the results of the other three subjects who were found negative in the conventional test for Helicobacter pylori, in which there are no obvious potential differences between the two data and the desired pH data substantially match up with the background pH data. It can thus be seen that the aforementioned method has high sensitivity and specificity for the detection of Helicobacter pylori in the stomach, and can quickly and accurately test whether the subject is infected with Helicobacter pylori.

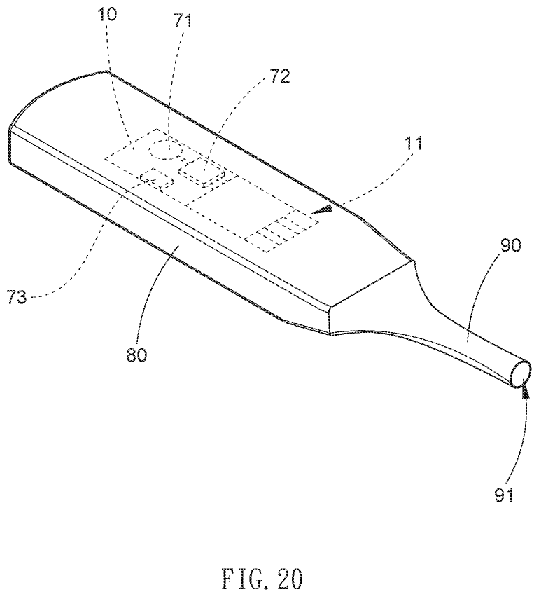

Please refer to FIG. 20 for a electrochemical test strip of the fifth embodiment of the present invention, in which the electrochemical test strip of the fifth embodiment in similar to that shown in FIGS. 6 and 7 with main differences in that the electrochemical test strip of the present embodiment further has a blowpipe 90 disposed on the casing 80, that the battery 71, the controller 72 and the antenna 73 are directly disposed on the strip body 10, and that the blowpipe 90 has a flow channel 91 for a gas sample to flow to the detection area 111.

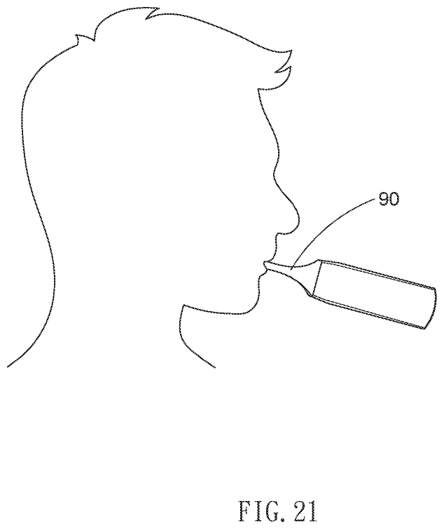

In the fifth embodiment of the present invention, a subject can blow on the blowpipe 90 as shown in FIG. 21 so that the detection area 111 is filled with the first gas sample blown by the subject. The electrochemical test strip can then detect the first gas sample and obtain a background pH data. The subject then consumes urea (e.g. carbon-12 urea) and waits for a period of time (e.g. more than 15 minutes), and then blow on the blowpipe of the same or a new electrochemical test strip so that the detection area is filled with the second gas sample blown by the subject. The electrochemical test strip then detects the second gas sample and obtain a desired pH data. The background pH data and the desired pH data can then be compared to determine whether the subject's stomach has Helicobacter pylori. During the sampling of the second gas sample, it is preferable to use a different, new electrochemicla test strip to avoid contamination of the detection area.

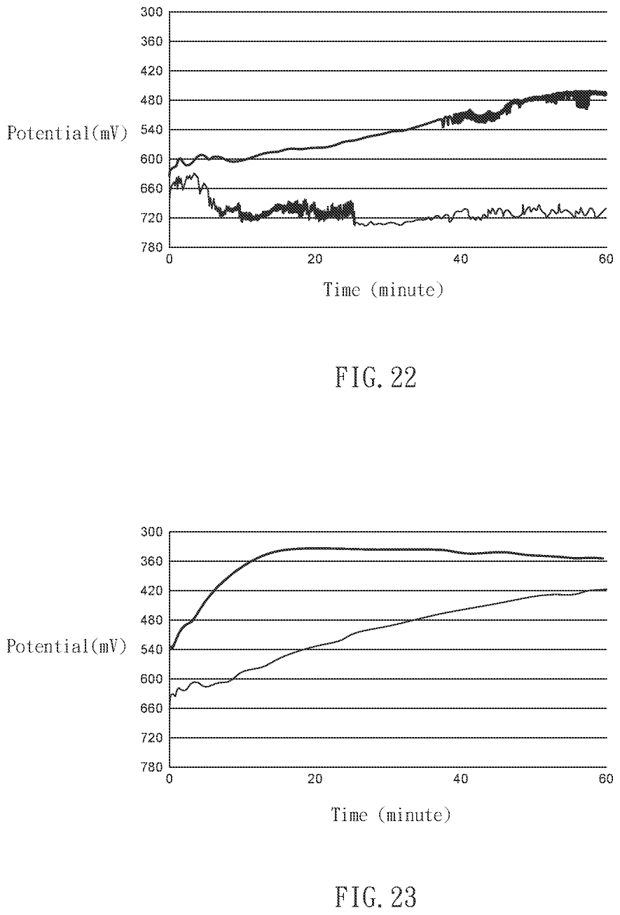

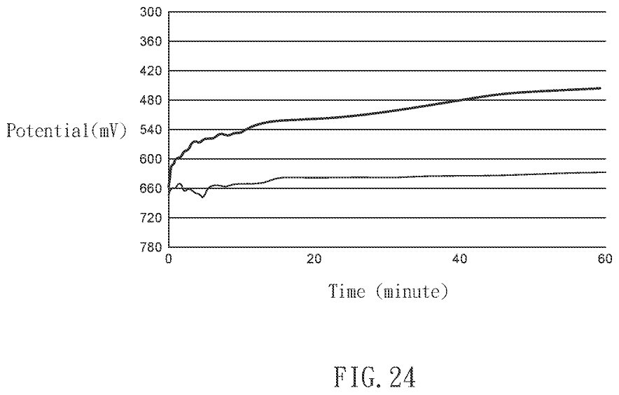

The applicant applied the method discussed in the fifth embodiment on three subjects to determine whether the subjects were infected with Helicobacter pylori. All three subjects were found positive in conventional tests for Helicobacter pylori. FIGS. 22-24 show the results of the aforementioned method performed by the three subjects, in which the thinner line represents a background pH data and the thicker line represents a desired pH data. It can be seen that there are obvious differences between the background pH data and the desired pH data and there are noticeable potential differences between the two data. Therefore, it can be confirmed that the method disclosed in the fifth embodiment can also quickly test whether the subject is infected with Helicobacter pylori.

What is claimed is:

1. A method for detecting Helicobacter pylori using an electrochemical test strip, wherein:
the electrochemical test strip comprises:
a strip body, having a detection area for contact with a gas sample;
a working electrode, disposed on the strip body and having a first part located in the detection area;
a pH sensing layer, disposed on the first part of the working electrode located in the detection area;
a reference electrode, disposed on the strip body and having a second part located in the detection area;
a solid water absorption layer, disposed in the detection area and covering the pH sensing layer and the second part, the solid water absorption layer being adapted to absorb or adsorb water in the gas sample in a manner that the first and second parts are electrically connected to each other;
a battery;
a controller, electrically connected to the working electrode, the reference electrode and the battery;
a casing covering the strip body; and
a blowpipe, disposed on the casing and having a flow channel for the gas sample to flow to the detection area;
the method for detecting Helicobacter pylori comprises the following steps:
filling the detection area with a first gas sample by making a subject blow on the blowpipe of the electrochemical test strip, and detecting the first gas sample with the electrochemical test strip to obtain a background pH data;
feeding the subject with urea;
filling the detection area with a second gas sample by making the subject blow on the blowpipe of the same or another said electrochemical test strip, and detecting the second gas sample with the electrochemical test strip to obtain a desired pH data; and
comparing the background pH data and the desired pH data to determine whether the subject's stomach has Helicobacter pylori.

2. The method for detecting Helicobacter pylori of claim 1, wherein the urea is carbon-12 urea.
